(19) United States
(12) Patent Application Publication
(10) Pub. No.: US 2012/0003317 A1
(43) Pub. Date: Jan. 5, 2012

(54) STABLE PROBIOTIC GRANULATE, AND METHOD FOR PREPARING SAME

(75) Inventors: Amelie Hiolle, Amfroipret (FR); Cecile Sampsonis, Bondues (FR); Eric Auclair, Avelin (FR); Christian Lenoir, Marquette Lez Lille (FR)

(73) Assignee: LESAFRE ET COMPAGNE, PARIS (FR)

(21) Appl. No.: 13/255,110

(22) PCT Filed: Mar. 9, 2010
(86) PCT No.: PCT/FR10/00193

§ 371 Date: Sep. 7, 2011

(30) Foreign Application Priority Data
Mar. 10, 2009 (FR) 0901094

(51) Int. Cl. A61K 9/14; A61P 1/00; A61K 35/66

(52) U.S. Cl. 424/489; 424/93.51; 424/93.3

(57) ABSTRACT

The invention pertains to the field of probiotic compositions useful for human and/or animal food and health. The invention particularly relates to compositions containing stable probiotic microorganisms during the production of tablets or granules for final use. The works of the applicant of the present invention show that the mortality of probiotic microorganisms during granulation is closely linked with granulation operating conditions and with the characteristics of the selected microorganisms. Said works have led to the establishment of a mathematical expression for defining both the characteristics of the yeast as well as the parameters of the method so that the microorganism losses after granulation are lower than 1 log CFU/g of granulated food.

The present invention relates to the field of probiotic compositions used for human and/or animal food and health. It relates particularly to a granulation method associated with a composition comprising probiotic microorganisms allowing the production of stable probiotic granules.

The term “probiotic” denotes live or viable microorganisms which, when they are ingested in sufficient quantity, exert a positive effect on the host organism by improving the properties of its intestinal flora beyond traditional nutritional effects. They constitute an alternative to the use of antibiotics in animal feed. They are most often bacteria or yeasts present either in foods or in food supplements.

There are four main types of probiotics:
lactic acid bacteria (lactobacilli and cocci),
bifidobacteria of human or animal origin,
various yeasts including yeasts of the Saccharomyces type,
other spore-forming bacteria, including Bacillus subtillis and cereus.

Microorganisms killed by heat do not meet the definition of probiotics even though it has been possible to attribute some therapeutic effects to them.

By extension, the term “probiotics” also denotes foods containing such microorganisms.

Probiotics are increasingly used both as human food and as animal feed, especially as substitutes for antibiotics given especially a return to a more healthy, more natural and more environmentally friendly diet. In animal nutrition, these microorganisms are in general added to a granular feed through a premixture also containing vitamins, trace elements and amino acids.

In general, the manufacture of granules is divided into at least two stages:
A—the preparation of an additive premixture,
B—the production, by the manufacturer, of food consisting of granules comprising the premixture (A).

The probiotic microorganisms are incorporated either into the premixture or during the manufacture of the granules.

The introduction of probiotic microorganisms into the granules is however not easy. The probiotic microorganisms are most often destroyed during the granulation process. Indeed, the manufacture of granules is carried out in the presence of steam at temperatures of between 60° C. and 120° C. and at pressure values ranging from 0.5 to 40 bar. These granulation methods combine very high thermal, mechanical and moisture stresses in which the microorganisms have to survive.

In patent application EP 0 694 610, the resistance to granulation of yeasts of the species Phaffia rhodozyma which produce a coloring, astaxanthin, and are used as fish food to give the red color to farmed salmon and trout, was improved by drying in the presence of a sugar such as glucose.

However, these were not probiotic yeasts which must remain live up to their ingestion by animals in order to fulfill their functions.

In patent application No. WO 2007/135278, it is stated that probiotic microorganisms could exhibit improved stability in premixtures over time by mixing with yeast walls and/or deactivated yeasts in place of other traditionally used premixture supports such as wheat flour or calcium carbonate, this improved stability over time then making them more resistant to granulation.

A real need therefore exists for probiotic microorganisms which are both stable over time and also during the granulation process such that the effect expected on the health of the one ingesting them is real.

The applicant has found that the selection of a probiotic microorganism for which the precise definition of the characteristics combined with the optimization of the operating conditions of the granulation method makes it possible to solve the problem cited above.

The solution of the invention allows better stability of the microorganisms, that is to say losses after treatment that are less than 1 log CFU/g (CFU: Colony Forming Unit).

More specifically, the applicant has found that the solution to this problem lies in the combination of the following two elements:
first of all, the selection of a microorganism chosen from the probiotic yeasts provided in a dry form agglomerated into spherules having a dry matter (DM) content of between 93 and 98% and a median diameter (d) of the spherules of between 500 and 1500 μm, and
next, the precise choice of the operating conditions for the granulation method used for the preparation of the granules intended for the final applications.

The solution forming the basis of the present invention does not generate losses of probiotic microorganisms after granulation that are greater than 1 log CFU/g of granular food as explained later.

The spherules according to the invention advantageously have a dry matter content of between 93 and 96% and a median diameter (d) of between 800 and 1200 μm.

In general, the production line for the granules is divided into several stages:
A—the preparation of an additive premixture (vitamins, trace elements, microorganisms, essential oils and the like, diluted on a support or adjuvant. There are several adjuvants, of mineral or plant origin, that are conventionally used such as wheat-based flour, calcium carbonate, sepiolite clay or residues from the starch industry). The premixture is provided in the form of a finely ground flour (median diameter of the particles of about 100 μm).
B—the production of the granules by the food manufacturer comprising:
a—a phase for macromixing with ground raw materials (wheat, corn, barley, oil cakes and the like), additives and premixtures. This macromixture is provided in the form of a coarse flour (median diameter of the particles of 500 μm),
b—a pre-granulation phase (heat treatment with steam alone or with mechanical stresses),
c—a granulation phase (manufacture of the granules) characterized by a die temperature (T) and a diameter (D) of the granules,
d—a drying-cooling phase.
Generally, the yeasts are incorporated into the premixture (stage A), or directly at the time of the macromixing (stage B).

The subject of the invention is a method for preparing probiotic granules comprising the following stages:
incorporation into a nutritional mixture suitable for the intended application of a probiotic composition (A) comprising:
   from 2 to 30% by weight of the total weight of the composition, of probiotic yeast having a dry matter (DM) content of between 93 and 98% and a median diameter (d) of the spherules of between 800 and 1200 μm, and
   from 70 to 98% by weight of the total weight of the composition, of a nutritional supplement comprising at least one constituent chosen from vitamins, trace elements, amino acids, and other additives intended for animal or human food and/or health,
injection of steam at a temperature of between 60° C. and 85° C. at pressure values ranging from 0.5 to 4 bar into the mixture obtained at the preceding stage,
granulation by pressing at a temperature of between 70 and 92° C. for the production of granules having a diameter (D) of between 2 and 6 mm, and
cooling.

The temperature (T) of the granulation die, the diameter of the granules (D), the dry matter (DM) content, and the median diameter (d) of the spherules are set so as to satisfy the following equation:

X=−196.482−0.023d+2.256(DM)−14.793T+3.046D+6.25.10⁻⁵d(DM)+0.001d.T+5.6310⁻⁵d.T+0.167(DM).T−0.036(DM).T−0.023D.T+4.06.10⁻⁶d²+0.003T²

in which the yeast losses after granulation×(log CFU/g initially−log CFU/g after granulation) are less than 1 log CFU/g of granular food.

According to a preferred embodiment, the method of the invention is carried out under the following conditions:

| | | | Dry matter content |
|---|---|---|---|
| Die temperature | Diameter of the | Diameter of the | of the spherules |
| (° C.) | granules (mm) | spherules (μm) | (%) |
| 85 | 3.5 | 1000 | 94.0 |
| | | 1200 | 93.0 |
| 90 | 4 | 800 | 94.0 |
| | | 970 | 94.8 |
| 92 | 4 | 865 | 94.8 |
| 92 | 4 | 950 | 94.7 |

The probiotic composition of the invention preferably comprises:
from 5 to 20% by weight of the total weight of the composition of probiotic yeast,
from 80 to 95% by weight of the total weight of the composition, of a nutritional supplement comprising at least one constituent chosen from vitamins, trace elements, amino acids, and other additives intended for animal or human food and/or health.

Yeast preparation and drying is a well known technique. The yeast is cultured in the form of a pure biomass, the yeast is then harvested, optionally mixed with technological aids in an amount of a few percent of the dry matter used, extruded and dried by various techniques: fluidization, drying on a belt or in a rotating drum. These various techniques are described in basic manuals such as Yeast Technology, Reed and Nagodawithana, second edition, an AVI book, Van Nostrand Reinhold. Persons skilled in the art know how to adapt the operating conditions, especially the dimensions of the granulation die and of the sieves, and the drying conditions so as to obtain the yeasts according to the present invention as indicated in the experimental section.

By way of example of a probiotic yeast, there may be mentioned the probiotic yeasts chosen from the group comprising yeasts of the genus Saccharomyces, preferably of the species Saccharomyces cerevisiae, or of the genus Kluyveromyces, such as Kluyveromyces marxianus, and mixtures thereof.

Preferred examples of Saccharomyces cerevisiae strains are the Saccharomyces cerevisiae Sc47 strain deposited at the NCYC under the number 47, the Saccharomyces cerevisiae strain deposited at the Pasteur collection (CNCM) under the number I-1077, the Saccharomyces cerevisiae strain deposited at the Pasteur collection (CNCM) under the number I-1079, the Saccharomyces cerevisiae strain deposited at the MUCL collection under the number 39 885, the Saccharomyces cerevisiae strain deposited at the CBS collection under the number 39 493.94, the Kluyveromyces marxianus strain deposited at the MUCL collection under the number 39434, and mixtures thereof.

Most preferably, the probiotic composition according to the present invention comprises yeasts corresponding to the Saccharomyces cerevisiae Sc47 strain deposited at the NCYC under the number 47.

The probiotic composition of the invention may be used as animal and/or human food. It may be administered in various forms.

According to a preferred embodiment of the invention, the probiotic composition intended as animal feed is administered in the form of granules.

According to a preferred embodiment of the invention, the following conditions are applied:

| | | | Dry matter |
|---|---|---|---|
| Die temperature | Diameter of the | Diameter of the | content of the |
| (° C.) | granules (mm) | spherules (μm) | spherules (%) |
| 85 | 3.5 | 1000 | 94.0 |
| | | 1200 | 93.0 |
| 90 | 4 | 800 | 94.0 |
| | | 970 | 94.8 |
| 92 | 4 | 865 | 94.8 |
| | | 950 | 94.7 |

The following conditions are those which cause the smallest losses:

| | | | Dry matter |
|---|---|---|---|
| Die temperature | Diameter of the | Diameter of the | content of the |
| (° C.) | granules (mm) | spherules (μm) | spherules (%) |
| 92 | 4 | 865 | 94.8 |
| 92 | 4 | 950 | 94.7 |

The composition (A) is introduced into the granules in an amount of 0.01 to 10% of probiotic yeast according to the invention relative to the dry weight of the food, which corresponds to 1.E7 to 1.E12 CFU per kg of granular foods.

Another subject of the invention is a granule comprising from 0.01 to 10% of probiotic yeast and from 90 to 99.9% by weight of a suitable nutritional mixture.

According to a preferred form, the granule comprises from 0.01% to 5% of probiotic yeast and from 95 to 99.9% of the nutritional mixture.

The granule of the invention is obtained with losses of probiotic microorganisms after granulation that are less than 1 log CFU/g of granular food.

Another subject of the invention is the use of the probiotic granule as animal and/or human food.

The text which follows describes the embodiments of the invention without limiting its scope.

The following studies were carried out:
identification of the critical granulation parameters for the yeasts,
study of the individual effects of the identified parameters on the survival of the yeast as a function of its physicochemical properties,
study of the total granulation on the yeast (as a pilot) with the aim of establishing a mathematical model linking the physical properties of the spherules and their stability,
determination of a pair [particle size and DM] leading to minimal losses,
validation of the pair [particle size and DM of the spherules] during industrial granulations.

In this work, the yeast in the ADY/SPH form was used. This is a dry bread yeast to be hydrated. It requires rehydration before use. It is provided in the form of spherules.

### I. Identification of the Critical Granulation Parameters for the Yeasts

Complex experimental designs made it possible to obtain the following classification of the critical parameters in decreasing order of severity: diameter of the granules, pre-granulation temperature, length of the channels of the die, moisture of the food. The physical constraints are therefore the moisture of the food, the treatment temperature and the compression (diameter and length of the channels being directly linked to the compression forces).

### II. Effect of the Total Granulation on the Stability of the ADY/SPH Yeasts

A complete experimental design (multifactorial, multilevel) varying the parameters below was followed:
parameters linked to the yeast: dry matter and median diameter
granulation parameters: die temperature, diameter of the granules.

The trials were carried out on a Kahl L14-175, 3 kW pilot press

| Factors | Levels | Values | | | |
|---|---|---|---|---|---|
| Dry matter (%) | 2 | 95 | 93 | | |
| Diameter of the | 4 | 1250 | 1050 | 865 | 570 |
| spherules (μm) | | | | | |
| Temperature (° C.) | 3 | 93 | 88 | 83 | |
| Diameter of the | 2 | 4 | 3.2 | | |
| granules (mm) | | | | | |

For example, there are a few results obtained:

| Median | | Diameter | | |
|---|---|---|---|---|
| diameter of the | | of the | | LOSSES |
| spherules | DM | granules | T ° C. | (log CFU/g) |
| 865 | 93 | 3.2 | 83 | 0.6 |
| 1250 | 95 | 3.2 | 83 | 0.5 |
| 1250 | 93 | 4 | 83 | 0.4 |
| 865 | 93 | 4 | 83 | 0.6 |
| 570 | 95 | 3.2 | 83 | 0.4 |
| 865 | 93 | 3.2 | 88 | 1.4 |
| 1250 | 95 | 3.2 | 93 | 1 |
| 1050 | 93 | 4 | 88 | 0.7 |
| 570 | 93 | 3.2 | 88 | 1.5 |
| 1250 | 95 | 3.2 | 88 | 1 |
| 1250 | 93 | 4 | 93 | 1.4 |
| 1050 | 95 | 3.2 | 93 | 0.6 |

This design led to the establishment of a model explaining the yeast losses after granulation as a function of these 4 factors.

The mathematical equation of the full model is presented below (valid in our experimental field).

X=−196.482−0.023d+2.256(DM)−14.793T+3.046D+6.25.10⁻⁵d(DM)+0.001d.T+5.6310⁻⁵d.T+0.167(DM).T−0.036(DM).T−0.023D.T+4.06.10⁻⁶d²+0.003T²
With:
   X represents the losses as microorganisms after granulation

(log CFU/g)=log CFU/g initially−log CFU/g
d (μm)=median diameter of the spherules
DM (%)=dry matter content of the spherules
D (mm)=diameter of the granules
T (° C.)=die temperature

According to our objectives for stability—losses that are less than 1 log CFU/g−the pairs [DM×median diameter of the spherules] which are of interest to us should be defined according to the granulation conditions applied. The mathematical equation then becomes:

1.0>−196.482−0.023d+2.256(DM)−14.793T+3.046D+6.25.10⁻⁵d(DM)+0.001d.T+5.6310⁻⁵d.T+0.167(DM).T−0.036(DM).T−0.023D.T+4.06.10⁻⁶d²+0.003T²

It is important to note that for given granulation conditions, there are a multitude of pairs [DM×median diameter of the spherules].

Here are a few examples of pairs which make it possible to have losses that are less than 1.0 log CFU/g of food:

| | | | Dry matter |
|---|---|---|---|
| Die temperature | Diameter of the | Diameter of the | content of the |
| (° C.) | granules (mm) | spherules (μm) | spherules (%) |
| 85 | 3.5 | 1000 | 94.0 |
| | | 1200 | 93.0 |
| 90 | 4 | 800 | 94.0 |
| | | 970 | 94.8 |
| 92 | 4 | 865 | 94.8 |
| | | 950 | 94.7 |

Two pairs were then selected for validation in industrial granulation (according to the feasibilities of manufacture of the products):

Pair 1: d=950 μm and DM=94.7%
Pair 2: d=865 μm and DM=94.8%
Detailed description of the method for preparing the yeast according to the invention

The yeast is prepared in the form of a pure biomass, it is then harvested, mixed with technological aids in an amount of a few percent of the dry matter used, extruded and dried by various techniques: fluidization, drying on a belt or in a rotating drum.

### A—Median Diameter (d):

A 2 mm extrusion mesh is used with the aim of obtaining spherules having a median diameter of 950 μm and a mesh of 1.8 mm in order to have spherules having a median diameter of 865 μm.

### B—Dry Matter Content (DM):

The method consists in drying in a conventional drum as described later. The drying times were optimized so as to have the desired DM content.

### Description of the Manufacturing Process

It is possible to distinguish several manufacturing stages, a dehydrating stage and a drying stage.

### A—Dehydration

This stage makes it possible to obtain a yeast paste containing 33% (plus or minus 3%) DM from a cream yeast containing 18% (plus or minus 2%) DM.

### Principle:

Brine (water+NaCl) is incorporated into the cream yeast until a conductivity varying between 15 000 μS and 20 000 μS is obtained.

This salted cream then passes onto a dehydrator (rotating filter under vacuum) on which a prelayer of potato starch (water+potato starch) will have been produced beforehand. After the passage onto the dehydrator, the cream yeast passes from a liquid state to a pasty state (30 to 36% DM).

This yeast paste is then crushed in a granulator (cylinder with mixing arms mixing the yeast and pushing it toward another cylinder at the end of which there is a perforated grid at 2 mm or 1.8 mm). This stage is crucial in the production of the desired particle size. The crushed yeast is then transported toward the drying organs.

### B—Drying

The drying is performed batchwise in a horizontal rotating cylinder provided with blades mixing the yeast and within which a hot air stream passes. The temperature and the flow rate of hot air are set so as to reach a yeast temperature of the order of 43° C. (+/−1° C.) at the end of drying.

Once the desired DM has been reached, the yeast is discharged into a silo for packaging.

1. (canceled)

2. (canceled)

3. (canceled)

4. (canceled)

5. (canceled)

6. (canceled)

7. (canceled)

8. (canceled)

9. (canceled)

10. (canceled)

11. A granulation method comprising the steps of:
a—incorporating, into a nutritional mixture suitable for an intended application, a probiotic composition (A) comprising:
from 2 to 30% by weight of the total weight of the composition, of a probiotic yeast in a dry form agglomerated into spherules having a dry matter (DM) content of between 93 and 98% and a spherule median diameter (d) of between 800 and 1200 m, and
from 70 to 98% by weight of the total weight of the composition, of a nutritional supplement comprising at least one constituent chosen from vitamins, trace elements, amino acids, and other additives intended for animal or human food and/or health,
b—injecting steam at a temperature of between 60° C. and 85° C. at pressure values ranging from 0.5 to 4 bar into the mixture obtained in step a,
c—granulating the mixture obtained in step b by pressing said mixture at a temperature of between 70° C. and 92° C. in order to produce granules having a diameter (D) of between 2 and 6 mm, and
d—cooling the granules obtained in step c,
wherein the temperature (T) of the granulating step, the diameter of the granules (D), the dry matter (DM) content, and the median diameter (d) of the spherules are set so as to satisfy the following equation:
X=−196.482−0.023d+2.256(DM)−14.793T+3.046D+6.25.10⁻⁵d(DM)+0.001d.T+5.63.10⁻⁵d.T+0.167(DM).T−0.036(DM).T−0.023D.T+4.06.10⁻⁶d²+0.003T²
wherein X is the yeast lost after granulation (log CFU/g initially−log CFU/g after granulation), and X is less than 1 log CFU/g of granular food.

12. The granulation method as claimed in claim 11, wherein the granulation temperature is 85° C., the diameter of the granules is 3.5 mm, the diameter of the spherules is 1000-1200 μm and the dry content of the spherules is 94.0-93.0%.

13. The granulation method as claimed in claim 11, wherein the granulation temperature is 90° C., the diameter of the granules is 4 mm, the diameter of the spherules is 800-970 μm and the dry content of the spherules is 94.0-94.8%.

14. The granulation method as claimed in claim 11, wherein the granulation temperature is 92° C., the diameter of the granules is 4 mm, the diameter of the spherules is 865 μm and the dry content of the spherules is 94.8%.

15. The granulation method as claimed in claim 11, wherein the granulation temperature is 92° C., the diameter of the granules is 4 mm, the diameter of the spherules is 950 μm and the dry content of the spherules is 94.7%.

16. The granulation method as claimed in claim 11, wherein the probiotic composition A comprises:
from 5 to 20% by weight of the total weight of the composition, of a probiotic yeast, and
from 80 to 95% by weight of the total weight of the composition, of a nutritional supplement comprising at least one constituent chosen from vitamins, trace elements, amino acids, and other additives intended for animal or human food and/or health.

17. The granulation method as claimed in claim 11, wherein the probiotic yeast is chosen from the group consisting of yeasts of the genus Saccharomyces, yeasts of the genus Kluyveromyces, and mixtures thereof.

18. The granulation method as claimed in claim 14, wherein the probiotic yeast of the genus Saccharomyces is a yeast of the species Saccharomyces cerevisiae, and the probiotic yeast of the genus Kluyveromyces is a yeast of the species Kluveromyces marxianus.

19. The granulation method as claimed in claim 15, wherein the probiotic yeast is selected from yeasts of strains chosen from the group consisting of the Saccharomyces cerevisiae Sc47 strain deposited at the NCYC under the number 47, the Saccharomyces cerevisiae strain deposited at the Pasteur collection (CNCM) under the number I-1077, the Saccharomyces cerevisiae strain deposited at the Pasteur collection (CNCM) under the number I-1079, the Saccharomyces cerevisiae strain deposited at the MUCL collection under the number 39 885, the Saccharomyces cerevisiae strain deposited at the CBS collection under the number 39 493.94, the Kluyveromyces marxianus strain deposited at the MUCL collection under the number 39434, and mixtures thereof.

20. The granulation method as claimed in claim 16, wherein the probiotic yeast is a yeast of the Saccharomyces cerevisiae Sc47 strain deposited at the NCYC under the number 47.

21. A probiotic granule obtained by the granulation method of claim 11, comprising:
from 0.01 to 5% by weight of a probiotic yeast, and
from 95 to 99.9% by weight of a suitable nutritional mixture.

22. The probiotic granule as claimed in claim 18, wherein the granule is used as animal feed.

23. The probiotic granule as claimed in claim 18, wherein the granule is used as human feed.
